(19) United States
(12) Patent Application Publication
(10) Pub. No.: US 2007/0265637 A1
(43) Pub. Date: Nov. 15, 2007

(54) DEVICES AND METHODS FOR CONTROLLING AND COUNTING INTERVENTIONAL ELEMENTS

(75) Inventors: Bernard H. Andreas, Redwood City, CA (US); Matthew McDonald, Santa Cruz, CA (US); Jay S. Daulton, Gilroy, CA (US); Chris Kilgus, Santa Cruz, CA (US); Andrew Leopold, Hawthorn Woods, IL (US); Philip Leopold, North Barrington, CA (US); Matt Maulding, Bellemont, AZ (US)

Correspondence Address: LEVINE BAGADE HAN LLP, 2483 EAST BAYSHORE ROAD, SUITE 100, PALO ALTO, CA, 94303, US

(73) Assignee: Xtent, Inc., Menlo Park, CA (US)

(21) Appl. No.: 11/735,392

(22) Filed: Apr. 13, 2007

Related U.S. Application Data
(60) Provisional application No. 60/745,373, filed on Apr. 21, 2006.

(51) Int. Cl. A61F 2/06

(52) U.S. Cl. 606/108; 623/1.11; 606/108

(57) ABSTRACT

Apparatus and methods for delivering stents or stent segments to body lumens include one or more tubular prostheses carried at the distal end of a catheter shaft, a sheath slidably disposed over the prostheses, and a guidewire tube extending from within the sheath to the exterior of the sheath through an exit port in a sidewall thereof. A guidewire extends slidably through the guidewire tube. The sheath can be moved relative to the catheter shaft and the guidewire tube to expose the prostheses for deployment. Mechanisms are described for measuring the distance that the sheath is moved relative to the catheter shaft and/or the guidewire tube, or for counting the number of stents exposed and/or deployed by operation of the device. These mechanisms include optical counting mechanisms, inductive, resistive, and/or magnetic resonating counters, electrical contact counters, and mechanical counters.

### CROSS-REFERENCE TO RELATED APPLICATIONS

The present application claims the benefit of priority to U.S. Provisional Patent Application Ser. No. 60/745,373 filed Apr. 21, 2006, which is incorporated herein by reference in its entirety.

### BACKGROUND OF THE INVENTION

The present invention relates generally to interventional catheters and prostheses, and more specifically to catheters and prostheses for treatment of vascular diseases, including coronary artery disease and peripheral vascular disease, as well as diseases of other body lumens such as the biliary tract, fallopian tubes, urinary and digestive tracts, and other structures.

Balloon angioplasty and stenting are widely used in the treatment of coronary artery disease and peripheral vascular disease. In coronary artery disease, one or more coronary blood vessels become narrowed or closed due to the buildup of stenotic plaques on the arterial wall. This blocks blood flow to the heart muscle, potentially causing myocardial infarction. Such narrowing can also occur in peripheral blood vessels such as the carotids, femorals, iliacs and other arteries, blocking the blood supply to other vital tissues and organs.

Balloon angioplasty involves the use of a long flexible catheter having a balloon at its distal tip. The catheter is inserted into a peripheral artery such as the femoral and advanced transluminally into the diseased artery. The balloon is inflated within the narrowed portion of the vessel, thereby expanding the vascular lumen and restoring normal blood flow.

In some cases, however, balloon angioplasty alone is inadequate to treat vascular disease due to restenosis, the renarrowing of the artery following angioplasty. Stents have been developed to provide an intravascular frame or scaffold to maintain patency of the vascular lumen after it has been expanded. Stents are small tubular prostheses designed to be advanced to the treatment site in a collapsed configuration using an elongated delivery catheter. The stents are then expanded at the treatment site into engagement with the vessel wall to maintain vascular patency.

Stents may be either self-expanding or balloon expandable. Self-expanding stents are made of a shape memory material such as Nitinol and can be delivered in a compressed state within the tip of the delivery catheter and allowed to resiliently expand upon release from the delivery catheter. Balloon expandable stents are made of a malleable metal and are mounted to a balloon on the delivery catheter. When positioned at the treatment site, the balloon is inflated to expand the stent into engagement with the vessel.

Stents, however, have also suffered from the problem of restenosis. Restenosis rates with conventional coronary stents have ranged from 30-40%. The causes of such restenosis are not fully understood. However, it is believed that restenosis may be caused in some cases by the excessive stiffness of current stents and their inability to conform to vascular curves, shapes, dimensional changes, and movements. This problem is particularly acute with longer lesions, which may extend over curved and tapered sections of a vessel and may be subject to non-uniform movements along their lengths.

The need has thus been demonstrated for highly flexible stents that may be used to treat long, curved, and tapered vascular regions. In co-pending U.S. patent application Ser. No. 10/637,713, filed Aug. 8, 2003, entitled “Apparatus and Methods for Delivery of Vascular Prostheses,” the full disclosure of which is incorporated herein by reference, highly flexible multi-segmented stents and associated delivery devices are disclosed that enable the treatment of long, curved or tapered vascular lesions. The disclosed delivery devices enable the selective deployment of one or more stent segments at a treatment site to allow the user to customize stent length in situ. Moreover, the device can be repositioned at multiple vascular sites to deploy a plurality of stents of various lengths.

Other custom-length stents and delivery devices are described in co-pending U.S. patent application Ser. No. 10/624,451, filed Jul. 21, 2003, entitled “Apparatus and Methods for Delivery of Multiple Distributed Stents,” which is also incorporated herein by reference. This application describes separable stent segments as well as continuous prosthesis structures configured as braids or coils that allow the user to pay out a selected length of the prosthesis structure and deploy it into the vessel at one or more treatment sites.

Variable length angioplasty devices have also been proposed. For example, U.S. Pat. No. 5,246,421 to Saab discloses angioplasty catheters having an elongated balloon and an external sheath that is axially slidable relative to the balloon. The sheath can be retracted to expose a selected length of the balloon for expansion at a treatment site. The catheter can then be repositioned and another length of balloon exposed to treat one or more additional sites.

While such custom-length stents and angioplasty catheters have shown great promise, there remains a need for improved ways of controlling and providing indication of balloon and stent length and/or number in such devices. Conventional angioplasty and stenting procedures rely upon the use of fluoroscopy to visualize the location and operation of catheters and prostheses. However, fluoroscopy often fails to provide the clarity, resolution, and precision that are required for the accurate control of stent or balloon length, which in many cases must be controlled within a few millimeters. Moreover, even if visualization were adequate, the user is left to control stent or balloon length by manually manipulating the associated catheters, an operation not well-suited to highly-precise control.

Devices for controlling and indicating the lengths of interventional elements such as balloons and stents are described in U.S. patent application Ser. No. 10/746,466, filed Dec. 23, 2003, entitled “Devices and Methods for Controlling and Indicating the Length of an Interventional Element,” which is also incorporated herein by reference. The devices for controlling the length of the interventional element described in the foregoing application include gear driven actuators, motors, and other mechanisms. The devices for indicating the length of the interventional element to the user described in the application include sensors, detents, visual displays, and other mechanisms providing visual, audible, and tangible indications of length. While these devices enable highly precise adjustment of the length of the interventional element deployed by the stent delivery catheter, there remains a need for delivery catheters that include more and/or improved mechanisms for providing an accurate count of stent segments deployed by the delivery catheter, and for providing accurate length information to the user.

For these and other reasons, stents and stent delivery catheters are needed which enable the customization of stent length in situ, and the treatment of multiple lesions of various sizes, without requiring removal of the delivery catheter from the patient. Such stents and stent delivery catheters should be capable of treating lesions of particularly long length and lesions in curved regions of a vessel, and should be highly flexible to conform to vessel shape and movement. Such stent delivery catheters should further be of minimal cross-sectional profile and should be highly flexible for endovascular positioning through tortuous vascular pathways.

### BRIEF SUMMARY OF THE INVENTION

The invention provides devices and methods for delivering prostheses or stents into body lumens and for indicating the length of and/or counting a number of prosthesis or stent segments on a medical device such as a catheter. The devices and methods facilitate accurate control of the working or deployed length of an interventional element, such as either a balloon, a stent, or other prosthesis, by providing mechanisms for accurately determining the deployed length of the balloon or stent(s), or for counting the number of stent segments exposed and/or deployed.

In one aspect of the invention, an apparatus for delivering a prosthesis into a target vessel comprises a flexible catheter shaft having proximal and distal ends and a first lumen therein. A tubular prosthesis is releasably carried near the distal end of the catheter shaft and is expandable to a shape suitable for engaging the target vessel. A sheath is disposed over the catheter shaft and the tubular prosthesis and is axially movable relative thereto. The sheath has proximal and distal ends, a sidewall, and an exit port in the sidewall between the proximal and distal ends. A guidewire tube extends through the exit port and has a distal extremity disposed within the tubular prosthesis and a proximal extremity disposed outside of the sheath, the guidewire tube being adapted for slidably receiving a guidewire therethrough.

The apparatus of the invention may be configured to deliver tubular prostheses that are either self-expanding or expandable by a balloon or other expandable member. When self-expanding prostheses are used, the sheath is adapted to constrain the prosthesis in a collapsed configuration. Upon retraction of the sheath, the prosthesis is released and self-expands to engage the vessel.

For balloon-expandable prostheses, an expandable member is mounted to the catheter shaft near the distal end thereof. The tubular prosthesis is positionable over the expandable member for expansion therewith. Usually the expandable member will comprise a balloon in communication with an inflation lumen in the catheter shaft for delivery of inflation fluid to the balloon. The sheath is axially positionable relative to the expandable member and configured to restrain expansion of a selected portion of the expandable member. Preferably the sheath is reinforced to prevent expansion thereof by the expandable member.

In a preferred aspect of the invention, the tubular prosthesis comprises a plurality of prosthesis segments. The sheath is axially movable relative to the prosthesis segments and configured to restrain expansion of a selectable number of prosthesis segments. In this way, lesions of various lengths may be treated by adjusting the length of the prosthesis in situ, without removal of the device from the body. In these embodiments, a pusher may be slidably disposed within the sheath proximal to the tubular prosthesis. The pusher has a distal end in engagement with the tubular prosthesis for moving the tubular prosthesis relative to the catheter shaft.

In a further aspect of the invention, a method of delivering a prosthesis in a target vessel of a patient comprises inserting a guidewire through the patient's vasculature to the target vessel; slidably coupling a delivery catheter to the guidewire, the delivery catheter having a sheath and a guidewire tube, a proximal extremity of the guidewire tube being outside the sheath and a distal extremity of the guidewire tube being inside the sheath, the guidewire being slidably positioned through the guidewire tube; advancing the delivery catheter over the guidewire to the target vessel; retracting the sheath relative to the guidewire tube to expose a tubular prosthesis carried by the delivery catheter; and expanding the tubular prosthesis into engagement with the target vessel.

In a preferred embodiment, an expandable member is fixed to a distal portion of the guidewire tube and the tubular prosthesis is positionable over the expandable member. The sheath is slidably disposed over the prosthesis and the expandable member and may be retracted a selectable distance to expose a desired length of the prosthesis and expandable member. The tubular prosthesis will then be expanded by expanding the expandable member. The sheath may be used to cover a proximal portion of the expandable member to constrain the proximal portion from expansion while a distal portion of the expandable member expands. Usually, the expandable member is inflatable and will be inflated by delivering inflation fluid to the expandable member through an inflation lumen in the catheter shaft. The guidewire tube preferably extends through the interior of the expandable member, which may be attached to the guidewire tube.

In a preferred aspect of the invention, the tubular prosthesis comprises a plurality of prosthesis segments, and the method includes positioning a first selected number of the prosthesis segments on the expandable member for expansion therewith. The method may further include positioning the sheath over a second selected number of the prosthesis segments to constrain expansion thereof. The first selected number of prosthesis segments may be positioned on the expandable member by pushing the first selected number with a pusher that is axially slidable relative to the expandable member.

In alternative embodiments, the tubular prosthesis self-expands when the sheath is retracted. In embodiments in which the prosthesis comprises multiple prosthesis segments, the sheath may be retracted relative to a selected number of such segments to allow the segments to self-expand into contact with the vessel.

In another aspect, the invention provides a balloon catheter for treating a target vessel that includes a flexible catheter shaft having proximal and distal ends and a first lumen therein. An expandable member is connected to the catheter shaft, and a sheath is disposed over the catheter shaft and the expandable member and is axially movable relative thereto. The sheath has an exit port in a sidewall thereof between its proximal and distal ends. A guidewire tube extends through the exit port and has a proximal extremity disposed outside of the sheath and a distal extremity disposed within the sheath that is coupled to the catheter shaft or the expandable member or both. The guidewire tube is adapted for slidably receiving a guidewire therethrough. The expandable member preferably comprises a balloon in fluid communication with the first lumen to receive inflation fluid therefrom. The sheath may be positionable to constrain a first selected portion of the expandable member from expansion while a second selected portion of the expandable member expands.

In a preferred embodiment of the balloon catheter of the invention, a tubular prosthesis is disposed on the expandable member and is expandable therewith. The tubular prosthesis will preferably comprise a plurality of unconnected stent segments that are slidable relative to the expandable member. The sheath is positionable to expose a first selected portion of the stent segments while covering a second selected portion of the stent segments.

In yet another aspect of the invention, an apparatus for delivering a prosthesis into a target vessel comprises a flexible catheter shaft having proximal and distal ends and a tubular prosthesis slidably coupled to the catheter shaft, the tubular prosthesis being expandable to a shape suitable for engaging the target vessel. A pusher is provided for moving the tubular prosthesis from a pre-deployment position to a deployment position near the distal end of the catheter shaft. The apparatus further includes a stop on the catheter shaft configured to engage the tubular prosthesis when the tubular prosthesis is in the deployment position.

In one embodiment, an expandable member is coupled to the catheter shaft and the tubular prosthesis is adapted for expansion by the expandable member. The expandable member, e.g. balloon, has an interior, and the stop is preferably disposed within the interior of the expandable member. The stop may also be disposed outside of or on the exterior surface of the expandable member. Alternatively, the tubular prosthesis is self-expanding and expands upon being released from the catheter shaft.

In a preferred aspect, a plurality of tubular prostheses are slidably coupled to the catheter shaft and are movable by the pusher to the deployment position. In addition, a sheath may be movably coupled to the catheter shaft and positionable over the tubular prosthesis or prostheses.

In a further method of deploying a tubular prosthesis in a target vessel according to the invention a catheter shaft is positioned in a target vessel and the tubular prosthesis is moved distally relative to the catheter shaft while the catheter shaft remains in the target vessel until the prosthesis engages a stop near the distal end of the catheter shaft. The tubular prosthesis is then expanded to engage a wall of the target vessel.

After expanding the tubular prosthesis, a second prosthesis (or any number of additional prostheses) may be moved distally relative to the catheter shaft until the second prosthesis engages the stop, and the second prosthesis then expanded to engage a wall of the target vessel. Alternatively, a second prosthesis may be moved distally relative to the catheter shaft simultaneously with moving the tubular prosthesis, and both the second prosthesis and the tubular prosthesis are expanded together to engage the wall of the target vessel. Usually, the tubular prosthesis and any additional prostheses are moved by a pusher movably coupled to the catheter shaft.

The tubular prosthesis is preferably expanded by inflating a balloon coupled to the catheter shaft. Alternatively, the tubular prosthesis may be self-expandable.

Further, the method may include retaining a second prosthesis in an unexpanded configuration on the catheter shaft while the tubular prosthesis is expanded. In one embodiment, the second prosthesis is retained within a sheath movably coupled to the catheter shaft.

In another aspect of the invention, an apparatus for delivering a prosthesis into a target vessel comprises a flexible catheter shaft having an inner shaft and an outer sheath slidable relative to the inner shaft. The flexible catheter shaft has proximal and distal ends and a tubular prosthesis slidably coupled to the catheter shaft, the tubular prosthesis being expandable to a shape suitable for engaging the target vessel. The tubular prosthesis is positioned over an expandable member, such as an inflation balloon, attached to the inner shaft. In several preferred embodiments, the tubular prosthesis comprises a plurality of stent segments. A pusher is provided for moving the tubular prosthesis from a pre-deployment position to a deployment position near the distal end of the catheter shaft.

The apparatus further includes a mechanism for determining the distance by which the outer sheath is retracted relative to the inner shaft during deployment of the tubular prosthesis. In several embodiments, the mechanism operates by detecting movement of a plurality of known reference points on the apparatus relative to a first fixed measuring point contained on the apparatus. Alternatively, the mechanism operates by counting the number of stent segments that are deployed during a stent deployment procedure. The measured parameter is able to be displayed to the user in a suitably useful format, such as a distance measurement, a listing of the counted number of stent segments, or the like.

In a first embodiment of the mechanism for determining outer sheath retraction distance, an optical fiber is attached to or embedded within one of the components of the flexible catheter, including either the outer sheath, the inner shaft, or the pusher. The optical fiber is provided with a transmission area that transmits a light beam and receives any signal reflected from a target that falls within the field of the transmission area. Suitable targets include reflective strips attached to other components of the catheter or the other components themselves. For example, when the optical fiber is attached to or embedded within the outer sheath, the light transmission area of the optical fiber may interact with individual stent segments, with portions of the pusher, or with reflective strips attached to either the inner shaft or the pusher. Alternatively, when the optical fiber is attached to or embedded within the inner shaft, the light transmission area of the optical fiber may interact with portions of the pusher, or with reflective strips attached to either the outer sheath or the pusher. In yet other embodiments, when the optical fiber is attached to or embedded within the pusher, the light transmission area of the optical fiber may interact with reflective strips attached to either the inner shaft or the outer sheath.

In another embodiment of the mechanism for determining outer sheath retraction distance, a sensor is attached to one of the components of the flexible catheter shaft at or near its distal end. The sensor is located and configured such that it will interact with another component of the catheter shaft, or with some other structure to detect the length by which the outer sheath has been moved relative to the inner shaft and/or relative to the balloon or the stent segments. One example of a suitable sensor is a resonating wire coil that is attached to or embedded within the outer sheath such that the wire coil surrounds the pusher. A lead wire extends from the wire coil to the proximal end of the catheter, where it is connected to a suitable user interface. As the outer sheath moves in relation to the pusher, the resonance amplitude of the wire coil changes, thereby changing a voltage carried by the wire coil. This voltage change may be measured and translated into a distance measurement or stent counter incrementation. Another example of a suitable sensor is a pressure sensor that may be attached to the internal surface of the outer sheath or the external surface of the pusher. A set of marker bands is included on the other, facing surface of the other component. As the outer sheath is moved relative to the pusher, the pressure sensor encounters the marker bands and experiences a change in pressure, which is measured and used to determine a distance or increment a stent counter. A suitable pressure sensor includes an elastomeric bump having a coating of a variable resistivity material, such as a variable resistivity ink. Still another example of a suitable sensor is a Hall Effect sensor that is mounted or attached to the outer sheath, the inner shaft, or the pusher. A plurality of suitable magnetic markers are attached to another of the catheter components such that the distance the outer sheath moves relative to the inner shaft or pusher may be determined and used to provide distance or count information to the user.

In still another embodiment of the mechanism for determining outer sheath retraction distance, a plurality of electrical conductors are attached to two or more of the components of the flexible catheter at or near its distal end, and are placed in contact with one another only when the outer sheath has moved a known distance relative to the inner shaft or the pusher. Accordingly, as a voltage is applied across the two conductors, the completion of a circuit indicates movement of the outer sheath over the known distance. A first example of this embodiment includes a first insulated electrical conductor attached to or embedded within the outer sheath, and having exposed sections at regular spaced intervals along the internal surface of the outer sheath. A particularly preferred example utilizes the reinforcing braid of the outer sheath as the first electrical conductor. The first electrical conductor engages spaced sections of the pusher at the spaced intervals along the length of the outer sheath, thereby closing a circuit at these locations. Accordingly, as a voltage is applied across the two conductors, the completion of a circuit indicates movement of the outer sheath over the known distance. A second example of this embodiment includes a first electrical lead attached to or embedded within the outer sheath and having an exposed portion located near the distal end of the outer sheath, such as, for example, at the location of the stent valve. Each stent segment is provided with an electrically conductive lead wire that is selectively retractable from the stent segment. The stent segment lead wires are in contact with the outer sheath lead wire when the particular stent segment is in a known location, such as at the location of the stent valve. Accordingly, the presence of a circuit between the outer sheath lead wire and the stent segment lead wire will indicate the position of the stent segments within the device.

In still another embodiment of the mechanism for determining outer sheath retraction distance, one or more position members are connected to one or more of the catheter components at or near the distal end(s) thereof. The proximal end(s) of the position member(s) are indexed such that the absolute and relative positions of the catheter component to which the position member is attached can be determined. Preferably, the position members comprise position wires that may be attached to the distal ends of one or more of the outer sheath, the inner shaft, and the pusher.

Further aspects of the nature and advantages of the invention will become apparent from the detailed description below taken in conjunction with the drawings.

### BRIEF DESCRIPTION OF THE DRAWINGS

FIG. 1 is a perspective view of a stent delivery catheter according to the invention with sheath retracted and expandable member inflated.

FIG. 2A is a side cross-section of a distal portion of the stent delivery catheter of FIG. 1 with expandable member deflated and sheath advanced distally.

FIG. 2B is a side cross-section of a distal portion of the stent delivery catheter of FIG. 1 with expandable member inflated and sheath retracted.

FIG. 3 is a transverse cross-section through line 3-3 of FIG. 2A.

FIG. 4 is a transverse cross-section through line 4-4 of FIG. 2A.

FIGS. 5A-5E are side cut-away views of the stent delivery catheter of the invention positioned in a vessel, illustrating various steps of delivering a prosthesis according to the method of the invention.

FIG. 6 is a side view of a pusher tube.

FIG. 6A is a cross-sectional view of the pusher tube of FIG. 6 taken at line AA.

FIG. 7A is a cross-sectional view of a generally distal portion of a stent delivery catheter illustrating an optical counter mechanism.

FIG. 7B is a cross-sectional view of a generally portion of a stent delivery catheter illustrating another optical counter mechanism.

FIG. 8 is a cross-sectional view of a generally distal portion of a stent delivery catheter illustrating still another optical counter mechanism.

FIG. 9 is a cross-sectional view of a generally distal portion of a stent delivery catheter illustrating a resonating coil counting mechanism.

FIG. 10 is a cross-sectional view of a generally distal portion of a stent delivery catheter illustrating an electrically conductive counting mechanism.

FIG. 11 is a cross-sectional view of a generally distal portion of a stent delivery catheter illustrating a counting mechanism that includes a sensor and a plurality of markers.

FIG. 12 is a cross-sectional view of a generally distal portion of a stent delivery catheter illustrating another electrically conductive counting mechanism.

FIG. 13 is a cross-sectional view of a generally distal portion of a stent delivery catheter illustrating still another electrically conductive counting mechanism.

FIG. 14 is a cross-sectional view of a generally distal portion of a stent delivery catheter illustrating a mechanical counting mechanism.

### DETAILED DESCRIPTION OF THE INVENTION

The present application relates generally to copending U.S. patent application Ser. No. 10/746,466, entitled “Devices and Methods for Controlling and Indicating the Length of an Interventional Element,” filed Dec. 23, 2003, which application is hereby incorporated by reference.

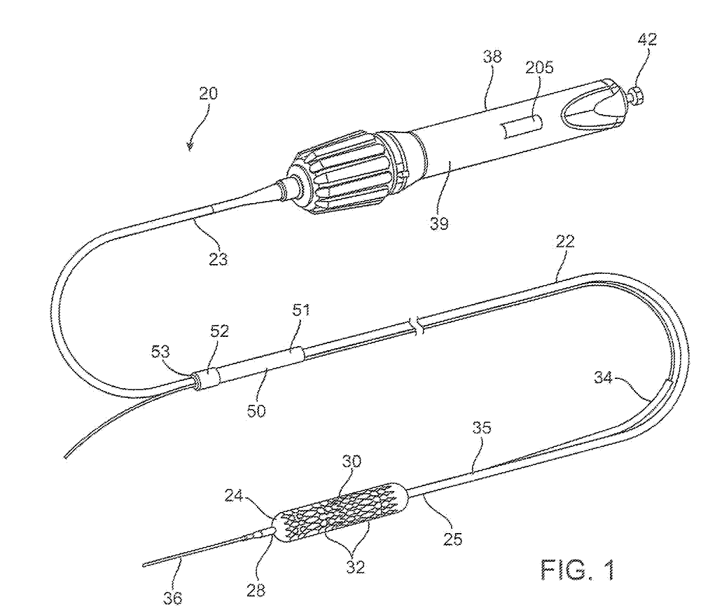

A first embodiment of a stent delivery catheter according to present invention is illustrated in FIG. 1. The stent delivery catheter 20 includes a catheter body 22 comprising an outer sheath 25 slidably disposed over an inner shaft 27 (not shown in FIG. 1). An expandable member 24, preferably an inflatable balloon (shown in an inflated configuration), is mounted to the inner shaft 27 and is exposed by retracting the sheath 25 relative to the inner shaft 27. A tapered nosecone 28, composed of a soft elastomeric material to reduce trauma to the vessel during advancement of the device, is mounted distally of expandable member 24. A stent 30, which preferably comprises a plurality of separate or separable stent segments 32, is disposed on the expandable member 24 for expansion therewith. A guidewire tube 34 is slidably positioned through a guidewire tube exit port 35 in the sheath 25 proximal to the expandable member 24. A guidewire 36 is positioned slidably through the guidewire tube 34, the expandable member 24, and the nosecone 28 and extends distally thereof.

Additional details of the construction, operation, and features of several preferred stent delivery catheters are described in co-pending U.S. Patent Application Ser. No. 60/688,896, filed Jun. 8, 2005, entitled “Apparatus and Methods for Deployment of Multiple Custom-Length Prostheses (P),” which application is hereby incorporated herein by reference. Embodiments of other preferred stent delivery catheters and details concerning their structure and operation are described in co-pending U.S. application Ser. No. 10/637,713, filed Aug. 8, 2003, entitled “Apparatus and Methods for Deployment of Vascular Prostheses,” which application is also hereby incorporated herein by reference.

A handle 38 is attached to a proximal end 23 of the sheath 25. The handle 38 performs several functions, including operating and controlling the catheter body 22 and the components included in the catheter body. Various embodiments of a preferred handle and additional details concerning its structure and operation are described in co-pending U.S. patent application Ser. No. 11/148,713, filed Jun. 8, 2005, entitled “Devices and Methods for Operating and Controlling Interventional Apparatus,” which application is hereby incorporated herein by reference. Embodiments of other preferred handles and details concerning their structure and operation are described in co-pending U.S. application Ser. No. 10/746,466, filed Dec. 23, 2003, entitled “Devices and Methods for Controlling and Indicating the Length of an Interventional Element,” which application is also hereby incorporated herein by reference.

The handle 38 includes a housing 39 that encloses the internal components of the handle. The inner shaft 27 is preferably fixed to the handle, while the outer sheath 25 is able to be retracted and advanced relative to the handle 38. An adaptor 42 is attached to the handle 38 at its proximal end, and is fluidly coupled to the inner shaft 27 in the interior of the housing of the handle 38. The adaptor 42 is configured to be fluidly coupled to an inflation device, which may be any commercially available balloon inflation device such as those sold under the trade name “Indeflator™”, available from Guidant Corp. of Santa Clara, Calif. The adaptor is in fluid communication with the expandable member 24 via an inflation lumen in the inner shaft 27 to enable inflation of the expandable member 24.

The outer sheath 25 and guidewire 36 each extend through a slider assembly 50 located on the catheter body 22 at a point between its proximal and distal ends. The slider assembly 50 is adapted for insertion into and sealing within a hemostatic valve, such as on an introducer sheath or guiding catheter, while allowing relative movement of the outer sheath 25 relative to slider assembly 50. The slider assembly 50 includes a slider tube 51, a slider body 52, and a slider cap 53.

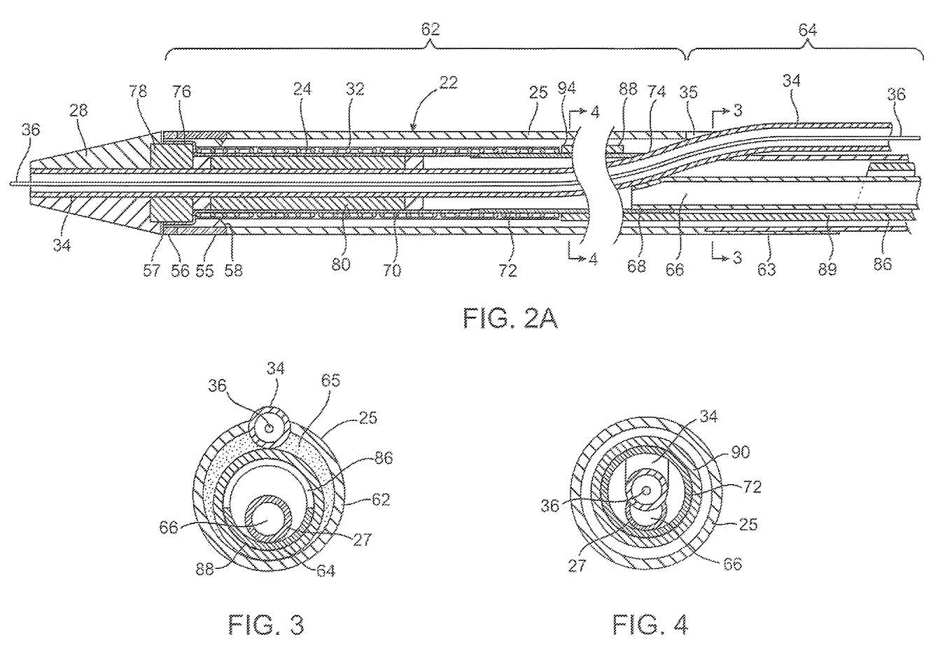

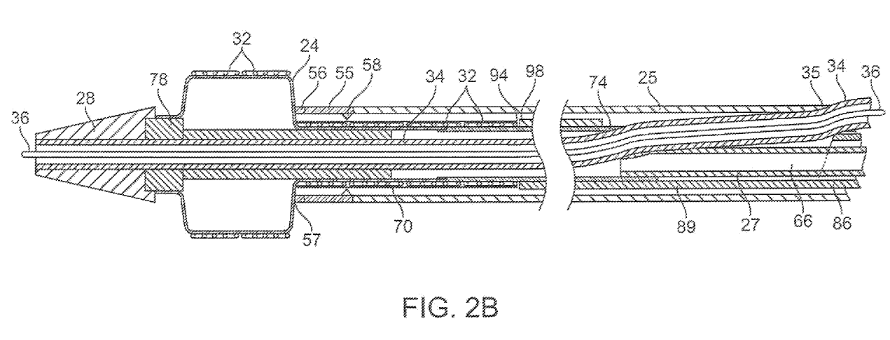

Referring now to FIGS. 2A-2B, 3 and 4, which show a distal portion of the stent delivery catheter in cross-section, it may be seen that the sheath 25 may be extended up to the nosecone 28 to fully surround the expandable member 24 and the stent segments 32. A garage 55 is attached to the outer sheath 25 at the distal end 57 of the sheath. The garage 55 is a generally cylindrical member having a relatively high circumferential strength such that it is able to prevent the expandable member 24 from inflating when the garage is extended over the inflatable member 24. The garage 55 preferably has a length at least as long as one of the stent segments 32 carried by the catheter, but preferably less than the combined length of two such stent segments. A radiopaque marker 56 is preferably formed integrally with or attached to the distal end of the garage 55 to facilitate visualization of the position of the sheath 25 using fluoroscopy. The radiopaque marker 56 may have an axial length selected to provide a visual reference for determining the appropriate distance for stent segment separation, e.g., 2-4 mm, as described below.

The outer sheath 25 further includes a valve member 58 within the garage 55 preferably spaced proximally from the distal end 57 a distance equal to, slightly larger than, or slightly smaller than the length of one of the stent segments 32. For example, in a preferred embodiment, each stent segment 32 has a length of about 4 mm, and the valve member 58 is located approximately 5 mm from the distal end 57 of the sheath or the distal end of the garage member 55. In other embodiments, the valve member 58 may be spaced from the distal end 57 a distance equal to about ¼-¾ of the length of one stent segment 32, more preferably one-half the length of one stent segment 32. The valve member 58 preferably comprises a necked-down circumferential waist or inwardly extending ring-shaped flange 60 configured to frictionally engage the stent segments 32 and thereby restrict the sliding movement of the stent segments 32 distally relative to the sheath 25. The flange 60 may be a polymeric or metallic material integrally formed with the sheath 25 or, preferably, with the garage 55, or a separate annular member bonded or otherwise mounted to the interior of the sheath 25 or the garage 55. The geometry of the flange 60 may be toroidal with a circular cross-section (like an O-ring) or it may have another cross-sectional shape such as triangular, trapezoidal, or pyramidal. Preferably, the flange 60 is a polymer such as silicone or urethane that is sufficiently soft, compliant, and resilient to provide frictional engagement with the stent segments 32 without damaging the stent segment or any coating deposited thereon. The valve member 58 will extend radially inwardly a sufficient distance to engage the exterior of the stent segments 32 with sufficient force to allow the line of stent segments 32 remaining within the sheath 25 to be retracted proximally with the sheath 25 so as to create spacing relative to those stent segments disposed distally of the sheath 25 for deployment. At the same time, the valve member 58 should not exert so much force that it removes or damages the coating on the exterior surface of the stent segments 32 as the sheath 25 is retracted relative to the stent segments to expose a desired number of stent segments 32. In a preferred embodiment, the stent segments 32 have an outer diameter of about 0.040-0.050 in. (including coating) and the sheath 25 and the garage 55 have inner diameter 0.041-0.051 in. so as to provide clearance of about 0.001 in. with the stent segments 32. The valve member 58 has a preferred inner diameter about 0.003-0.008 in. less than that of the garage 55, or about 0.033-0.048″, so as to provide an interference fit with the stent segments 32. The valve member 58 will preferably exert a force of about 0.5-5 lbs. on a stent segment 32 positioned within it. Various embodiments of the valve member 58 are described in copending application Ser. No. 10/412,714, Filed Apr. 10, 2003, which is incorporated herein by reference.

As thus described, the sheath 25 has a distal extremity 62 configured to surround the expandable member 24 and the stent segments 32 disposed thereon when in an unexpanded configuration. The distal extremity 62 extends proximally to a junction 63, preferably aligned with the location of the guidewire tube exit port 35, where the distal extremity 62 is joined to a proximal extremity 64 that extends proximally to the handle 38 (see FIG. 1). In a preferred embodiment, the distal extremity 62 has a length of about 15-35 cm and the proximal extremity 64 as a length of about 100-125 cm. The proximal extremity 64 may be constructed of a variety of biocompatible polymers, metals, or polymer/metal composites, preferably being stainless steel or Nitinol. The distal extremity 62 may be a polymer such as PTFE, FEP, polyimide, nylon, or Pebax, or combinations of any of these materials. In a preferred form, the distal extremity 62 comprises a composite of nylon, PTFE, and polyimide. The distal extremity is preferably reinforced with a metallic or polymeric braid to resist radial expansion when expandable member 24 is expanded. The sheath 25 may further have a liner surrounding its interior of low friction material such as PTFE to facilitate relative motion of the sheath 25, the stent segments 32, and the pusher tube 86.

Preferably, the proximal extremity 64 has a smaller transverse dimension than the distal extremity 62 to accommodate the added width of the guidewire tube 34 within the vessel lumen, as well as to maximize flexibility and to minimize profile. In one embodiment, shown in FIG. 3, the distal extremity 62 is a tubular member having a first outer diameter, preferably about 1.0-1.5 mm, and the proximal extremity 64 is a tubular member having a second, smaller outer diameter, preferably about 0.7-1.0 mm. At the junction of the proximal extremity 64 with the distal extremity 62, a proximally-facing crescent-shaped opening 65 is formed between the two tubular members that creates the guidewire tube exit port 35. Excess space within the crescent-shaped opening 65 may be filled with a filler material such as adhesive or a polymeric material (e.g., Pebax).

The guidewire tube 34 is slidably positioned through the guidewire tube exit port 35. The guidewire tube exit port 35 may be configured to provide a total or partial fluid seal around the periphery of the guidewire tube 34 to limit blood flow into the interior of the sheath 25 and to limit leakage of saline (or other flushing fluid) out of the sheath 25. This may be accomplished by sizing the guidewire tube exit port 35 appropriately so as to form a fairly tight frictional seal around the guidewire tube 34 while still allowing the sliding motion thereof relative to the sheath 25. Alternatively, an annular sealing ring may be mounted in the guidewire tube exit port 35 to provide the desired seal. Preferably, however, the guidewire tube exit port 35 is not totally fluid sealed, so as to provide a slight leakage or fluid flow to provide the ability to flush the distal extremity 62 of the catheter.

The guidewire tube exit port 35 will be positioned to provide optimal tracking of the stent delivery catheter 20 through the vasculature and maximizing the ease with which the catheter can be inserted onto and removed from a guidewire to facilitate catheter exchanges. Usually, the guidewire tube exit port 35 will be positioned at a location proximal to the expandable member 24 when the sheath 25 is extended fully distally up to the nosecone 28, but a distance of no more than one-half the length of the sheath 25 from the distal end 57. In preferred embodiments for coronary applications, the guidewire tube exit port 35 is spaced proximally a distance of about 20-35 cm from the distal end 57 of the sheath 25.

The guidewire tube 34 should extend proximally from the guidewire tube exit port 35 a distance at least as long as the longest possible stent that may be deployed, e.g., 30-200 mm depending upon the application, to allow for retraction of the sheath 25 that distance while retaining a portion of the guidewire tube 34 external to the sheath 25. Preferably the guidewire tube 34 extends proximally a distance of about 35 to about 70 mm from the guidewire tube exit port 35 when the sheath 25 is in a fully distal position, with the proximal end thereof disposed a distance of about 23-50 cm from the distal tip of the nosecone 28. In applications in which the stent delivery catheter 20 is to be positioned through a guiding catheter, the proximal end of the guidewire tube 34 will preferably be positioned so as to be within the guiding catheter when the expandable member 24 is positioned at the target site for stent deployment. The guidewire tube 34 is preferably a highly flexible polymer such as PTFE, FEP, polyimide, or Pebax, and may optionally have a metal or polymer braid or fiber embedded in it to increase kink-resistance and tensile strength.

The inner shaft 27 forms an inflation lumen 66 that is in communication with the interior of the expandable member 24. The inner shaft 27 may be formed of a polymer material such as PTFE, FEP, polyimide, or Pebax, or the inner shaft 27 may be a metal such as stainless steel or Nitinol.

The expandable member 24 has an expandable balloon member 70 that is joined to a non-expandable tubular leg 72. The expandable balloon member 70 is a semi-compliant polymer such as Pebax, polyurethane, or Nylon. Non-compliant, fully elastic, or other materials such as PTFE may also be used. Preferably, the compliance of the balloon member allows the expanded diameter of the balloon member 70 to be adjusted by selecting the appropriate inflation pressure delivered thereto, thereby allowing customization of the deployed diameter of stent segments 32. For example, in one embodiment, the balloon member 70 may be inflated to a pressure of between about 5 and about 12 atmospheres, allowing the deployed stent diameter to be adjusted from about 2.0 mm to 4.0 mm. Of course, larger and smaller stent diameters are also possible by utilizing appropriate stent geometry and applying suitable inflation pressures.

The tubular leg 72 is preferably a polymer such as polyimide, PTFE, FEP, polyurethane, or Pebax and may optionally be reinforced with a metal or polymer braid or metal or polymer fibers. The tubular leg 72 has an open proximal end 74 through which the guidewire tube 34 extends. The proximal end 74 of the tubular leg 72 is fixed to the distal end 68 of the inner shaft 27 and to the guidewire tube 34, forming a fluid-tight seal. The guidewire tube 34 passes through the interior of the balloon member 70 and is mounted to the nosecone 28, thereby providing a passage through the distal portion of the catheter body 22 through which the guidewire 36 may pass. The balloon member 70 has a distal end 76 that extends over an annular stop 78, which is mounted to the distal end of the guidewire tube 34 and/or the nosecone 28. The distal end 76 of the balloon member 70 may be bonded to the stop 78, the guidewire tube 34, and/or the nosecone 28. The stop 78 has a size and shape selected to engage the stent segment 32 and provide a stop against which the stent segments 32, can be located in the ideal deployment position without being pushed beyond the distal end of the balloon member 70. Additional details concerning stent stops suitable for use in the devices and methods described herein are disclosed in U.S. patent application Ser. No. 10/884,616, filed Jul. 2, 2004, which is hereby incorporated by reference herein.

Optionally, within the interior of the balloon member 70 an annular base member 80 is mounted to the guidewire tube 34 and has a diameter selected to urge the balloon member 70 against the stent segments 32 in their unexpanded configuration, thereby providing frictional engagement with the stent segments 32. This helps to limit unintended sliding movement of the stent segments 32 on the balloon member 70. The base member 80 may be made of a soft elastomer, foam, or other compressible material.

The stent segments 32 are slidably positioned over the balloon member 70. Depending upon the number of stent segments 32 loaded in the stent delivery catheter 20, the stent segments 32 may be positioned over both the balloon member 70 and the tubular leg 72. In an exemplary embodiment, each stent segment is about 2-20 mm in length, more preferably 2-8 mm in length, and 3-50 stent segments may be positioned end-to-end in a line over the balloon member 70 and the tubular leg 72. The stent segments 32 preferably are in direct contact with each other, but alternatively separate spacing elements may be disposed between adjacent stent segments, the spacing elements being movable with the stent segments along the balloon member 70.

The stent segments 32 are preferably a malleable metal so as to be plastically deformable by the expandable member 24 as they are expanded to the desired diameter in the vessel. Alternatively, the stent segments 32 may be formed of an elastic or super elastic shape memory material such as Nitinol so as to self-expand upon release into the vessel by retraction of sheath 25. The stent segments 32 may also be composed of polymers or other suitable biocompatible materials including bioabsorbable or bioerodable materials. In self-expanding embodiments, the expandable member 24 may be eliminated or may be used for predilatation of a lesion prior to stent deployment or for augmenting the expansion of the self-expanding stent segments.

In preferred embodiments, the stent segments 32 are coated with a drug that inhibits restenosis, such as Rapamycin, Paclitaxel, Biolimus A9 (available from BioSensors International), analogs, prodrugs, or derivatives of the foregoing, or other suitable agent, preferably carried in a durable or bioerodable polymeric or other suitable carrier material. Alternatively, the stent segments 32 may be coated with other types of drugs and therapeutic materials such as antibiotics, thrombolytics, anti-thrombotics, anti-inflammatories, cytotoxic agents, antiproliferative agents, vasodilators, gene therapy agents, radioactive agents, immunosuppressants, and chemotherapeutics. Several preferred therapeutic materials are described in U.S. Published Patent Application No. 2005/0038505, entitled “Drug-Delivery Endovascular Stent and Method of Forming the Same,” filed Sep. 20, 2004, which application is hereby incorporated by reference herein. Such materials may be coated over all or a portion of the surface of the stent segments 32, or the stent segments 32 may include apertures, holes, channels, pores, or other features in which such materials may be deposited. Methods for coating stent segments 32 are described in the foregoing published patent application. Various other coating methods known in the art may also be used, including syringe application, spraying, dipping, inkjet printing-type technology, and the like.

The stent segments 32 may have a variety of configurations, including those described in copending U.S. Patent Application Ser. No. 60/688,896, filed Jun. 8, 2005, and Ser. No. 10/738,666, filed Dec. 16, 2003, each of which is incorporated herein by reference. The stent segments 32 are preferably completely separate from one another without any interconnections, but alternatively may have couplings between two or more adjacent segments which permit flexion between the segments. As a further alternative, one or more adjacent stent segments may be connected by separable or frangible couplings that are separated prior to or upon deployment, as described in co-pending U.S. patent application Ser. No. 10/306,813, filed Nov. 27, 2002, which is also incorporated herein by reference.

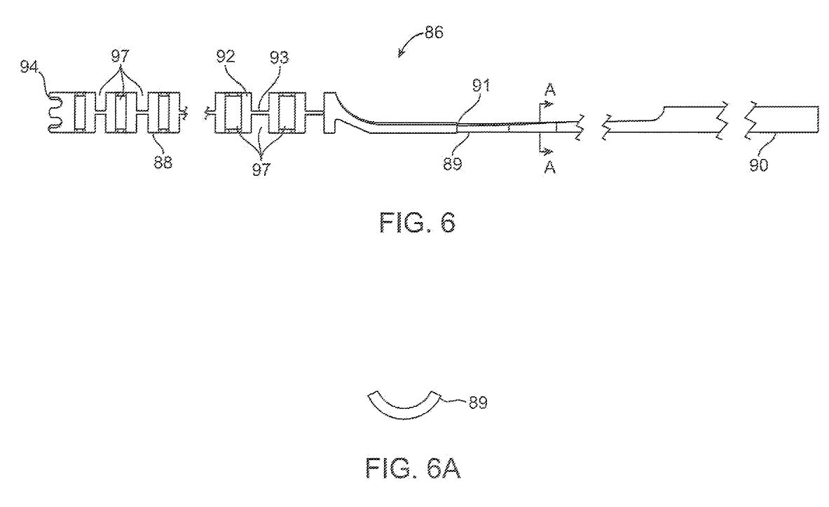

A pusher tube 86 is slidably disposed over the inner shaft 27. The structure of the pusher tube 86 is illustrated in FIG. 6, and its location within the catheter body 22 is best shown in FIGS. 2A-B. The pusher tube 86, contains three primary sections, a distal extension 88, a ribbon portion 89, and a proximal portion 90. The proximal portion 90 extends from the handle 38 over the inner shaft 27 and to the ribbon portion 89. The proximal portion 90 is preferably formed of a tubular material to provide high column strength but adequate flexibility to extend through the vasculature from an access site to the coronary ostia or other target vascular region. A preferred material is stainless steel hypotube. The ribbon portion 89 of the pusher tube corresponds with the location of the guidewire exit port 35 on the outer sheath 25. The ribbon portion 89 is formed of a partial-tube, see, e.g., FIG. 6A, in order to provide an opening to allow the guidewire tube 34 to pass through to the exit port 35. The proximal portion of the ribbon portion 89 is formed out of the same tubular material that makes up the proximal portion 90 of the pusher tube, e.g., stainless steel hypotube.

The proximal portion of the ribbon portion 89 is joined to the distal portion of the ribbon 89, such as by a weld 91 or the ribbon portion and proximal portion may be formed from the same hypotube which is laser cut in the appropriate geometry. The distal extension 88 is preferably formed of a slotted tube of rigid material, such as stainless steel or Nitinol. The slotted tube making up the distal extension 88 includes a number of cylindrical rings 92 interconnected by longitudinal connectors 93, thereby defining a plurality of transverse slots 97 arranged in pairs along the length of the distal extension. Each pair of slots is disposed opposite one another on distal extension 88, thus defining a pair of opposing, longitudinal connectors 93. The longitudinal connectors 93 are flexible so as to be capable of bending around a transverse axis. Each pair of transverse slots 97 is oriented at 90 degrees relative to the adjacent pair of slots 97, so that the pairs of longitudinal connectors 93 alternate between those oriented vertically and those oriented horizontally. This allows distal extension 88 to bend about either a horizontal and vertical transverse axes, thus providing a high degree of flexibility. Of course, the pairs of transverse slots 97 could be oriented at various angles relative to adjacent pairs to provide flexibility about more than two axes. The slots provided in the slotted tube allows the distal extension 88 to be more axially flexible than it would be without the slots, while still retaining high column strength. It is preferable to provide transverse slots 97 and cylindrical rings 92 that each have a width that is approximately the same as the length of a stent segment 32. In addition or alternatively, the transverse slots 97 and cylindrical rings 92 may be spaced apart by a known fraction or multiple of the stent segment length. In this way, a detent mechanism may be provided on the interior surface of the sheath 25, with one or more detents that releasably engage the cylindrical rings 92 formed in the distal extension 88 to provide a tactile feedback based upon the distance that the outer sheath 25 is retracted relative to pusher tube 86.

A nesting tip 94 is formed on the distal end of the distal extension 88. The nesting tip preferably includes a plurality of fingers shaped and oriented to engage and interleave with the proximal end of the most proximal stent segment 32. The stent segments 32 preferably have axial extensions or projections on each end which interleave with those on the adjacent stent segment. The tip 94 of pusher tube 86 preferably has a geometry with axial projections similar to or complementary to those of the stent segments 32 so as to interleave therewith.

Preferably, the proximal portion 90 of the pusher tube has a diameter that is smaller than the diameter of the distal extension 88. Thus, the stainless steel hypotube material making up the proximal portion 90 of the pusher tube and part of the ribbon portion 89 may have a first diameter, while the slotted tube making up the distal extension 88 and the distal portion of the ribbon 89 may have a second, larger diameter. As noted above, the slotted tube and the hypotube are preferably joined by a weld 91 formed in the ribbon portion 89.

As best shown in FIGS. 2A-B, the pusher tube 86 extends longitudinally within the outer sheath 25 and over the inner shaft 27 through most of the length of the catheter body 22. The distal extension 88 is slidable over the tubular leg 72 and engages the stent segment 32 at the proximal end of the line of stent segments 32. At its proximal end (not shown), the pusher tube 86 is coupled to an actuator associated with the handle 38 (see FIG. 1). In this way, the pusher tube 86 can be advanced distally relative to the inner shaft 27 to urge the stent segments 32 distally over the expandable member 24 (or, alternatively, the pusher tube 86 may be held in position while retracting the expandable member 24 relative to stent segments 32) until the stent segments engage the stop 78. In addition, the pusher tube 86 can be used to hold the stent segments 32 in place on the expandable member 24 while the sheath 25 is retracted to expose a desired number of stent segments 32, as shown in FIG. 2B. As noted above, the proximal portion 90, ribbon portion 89, and distal extension 88 of the pusher tube are preferably constructed of stainless steel, but they may alternatively be constructed of a variety of biocompatible polymers, metals, polymer/metal composites, alloys, or the like.

It can be seen that with the sheath 25 retracted a desired distance, the expandable member 24 is allowed to expand when inflation fluid is delivered through the inflation lumen 66, thereby expanding a desired number of stent segments 32 exposed distally of sheath 25. The remaining portion of the expandable member 24 and the remaining stent segments 32 within sheath 25 are constrained from expansion by the sheath 25.

FIG. 2B further illustrates that when the sheath 25 is retracted relative to the expandable member 24, the guidewire tube exit port 35 becomes further away from the point at which the guidewire 36 exits the proximal end 74 of the tubular leg 72, increasing the distance that the guidewire 36 must pass within the interior of the sheath 25. Advantageously, the guidewire tube 34 provides a smooth and continuous passage from the tubular leg 72 through the guidewire tube exit port 35, eliminating any problems that might result from changing the alignment of the two. This is particularly important in the present device where the stent delivery catheter may carry a large number of stent segments 32 and the sheath 25 may be retracted a substantial distance relative to the expandable member 24, resulting in substantial misalignment of the guidewire tube exit port 35 relative to the tubular leg 72.

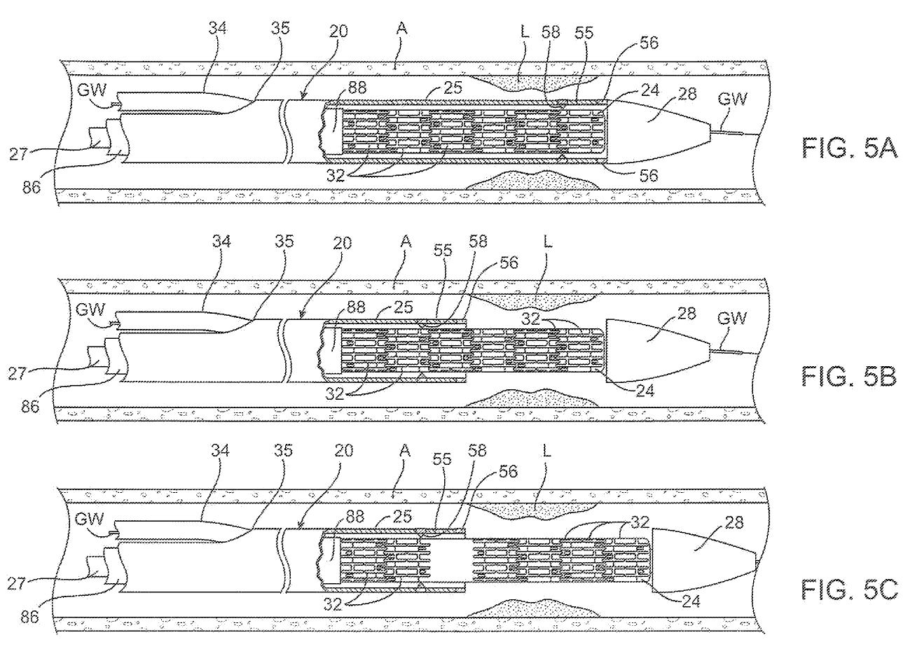

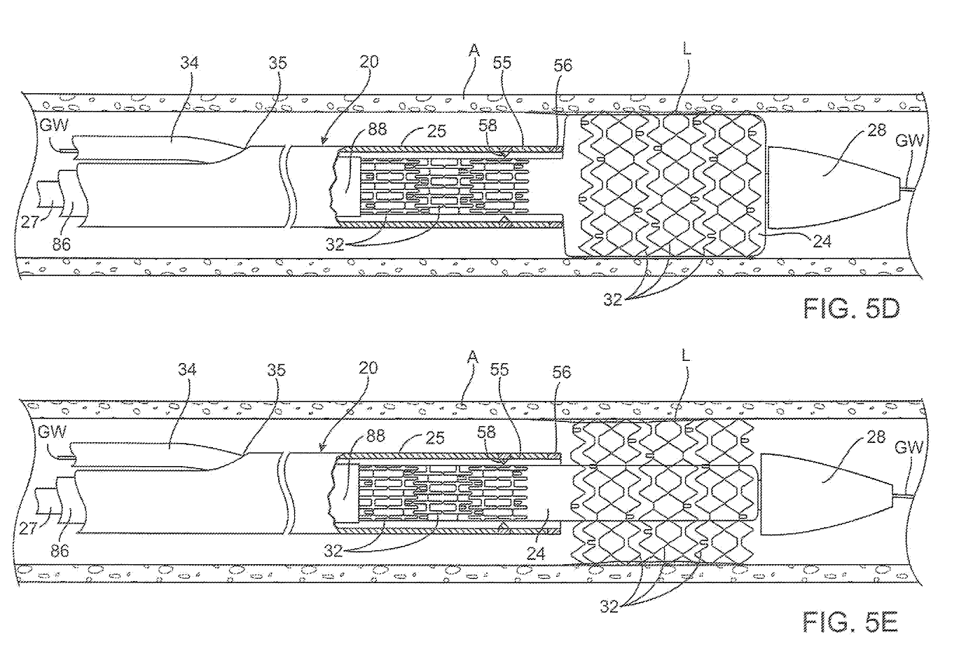

Referring now to FIGS. 5A-5E, the use of the stent delivery catheter of the invention will be described. While the device will be described in the context of coronary artery treatment, it should be understood that the device is useful in any of a variety of blood vessels and other body lumens in which stents are deployed, including the carotid, femoral, iliac and other arteries, as well as veins and other fluid-carrying vessels. A guiding catheter (not shown) is first inserted into a peripheral artery such as the femoral and advanced to the ostium of the target coronary artery. A guidewire GW is then inserted through the guiding catheter into the coronary artery A where lesion L is to be treated. The proximal end of guidewire GW is then inserted through the nosecone 28 and the guidewire tube 34 outside the patient's body and the stent delivery catheter 20 is slidably advanced over the guidewire GW and through the guiding catheter into the coronary artery A. The slider assembly 50 is positioned within the hemostasis valve at the proximal end of the guiding catheter, which is then tightened to provide a hemostatic seal with the exterior of the slider body 52. The stent delivery catheter 20 is positioned through a lesion L to be treated such that the nosecone 28 is distal to the lesion L. During this positioning, the sheath 25 is positioned distally up to the nosecone 28 so as to surround the expandable member 24 and all of the stent segments 32 thereon.

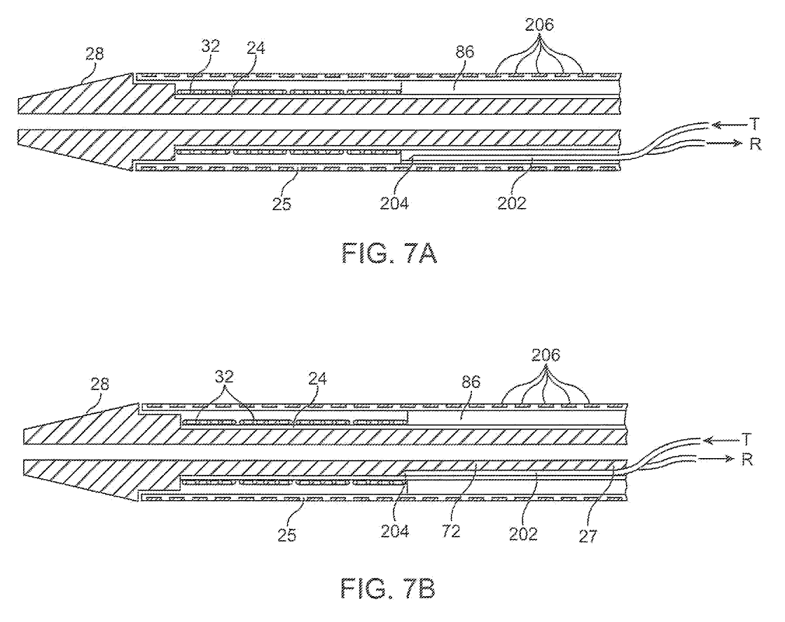

Optionally, the lesion L may be pre-dilated prior to stent deployment. Pre-dilation may be performed prior to introduction of the stent delivery catheter 20 by inserting an angioplasty catheter over the guidewire GW and dilating the lesion L. Alternatively, the stent delivery catheter 20 may be used for pre-dilation by retracting the sheath 25 along with the stent segments 32 to expose an extremity of the expandable member 24 long enough to extend through the entire lesion. This may be done while the delivery catheter 20 is positioned proximally of the lesion L or with the expandable member 24 extending through the lesion L. Fluoroscopy enables the user to visualize the extent of the sheath retraction relative to the lesion L by observing the position of the marker 56 on the garage 55 contained at the distal end of the sheath 25 relative to markers that may be formed on or attached to the guidewire tube 34 beneath the expandable member 24. Alternatively, the extent of sheath retraction may be detected by one of the mechanisms described below in relation to FIGS. 7-14. To allow the stent segments 32 to move proximally relative to the expandable member 24, force is released from the pusher tube 86 and the valve member 58 engages and draws the stent segments proximally with the sheath 25. The pusher tube 86 is retracted along with the outer sheath 25 by use of an actuator provided on the handle 38. With the appropriate length of the expandable member 24 exposed, the expandable member 24 is positioned within the lesion L and inflation fluid is introduced through the inflation lumen 66 to inflate the expandable member 24 distally of the sheath 25 and thereby dilate the lesion L. The expandable member 24 is then deflated and retracted within the sheath 25 while maintaining force on the pusher tube 86 so that the stent segments 32 are positioned up to the distal end of the expandable member 24, surrounded by the sheath 25.

Following any predilatation, the stent delivery catheter 20 is repositioned in the artery A so that the nosecone 28 is distal to the lesion L as shown in FIG. 5A. The sheath 25 is then retracted as in FIG. 5B to expose the appropriate number of stent segments 32 to cover the lesion L. Again, fluoroscopy can be used to visualize the position of the sheath 25 by observing the marker 56 thereon relative to a marker 82 within the expandable member 24. As the sheath 25 is drawn proximally, force is maintained against the pusher tube 86 so that the stent segments 32 remain positioned up to the distal end of the expandable member 24. It should also be noted that the sheath 25 moves proximally relative to the guidewire tube 34, which slides through the guidewire tube exit port 35. Advantageously, regardless of the position of the sheath 25, the guidewire tube 34 provides a smooth and continuous passage for the guidewire GW so that the stent delivery catheter slides easily over the guidewire GW.

With the desired number of stent segments 32 exposed distally of the sheath 25, it is preferable to create some spacing between the stent segments to be deployed and those remaining enclosed within the sheath 25. This reduces the risk of dislodging or partially expanding the distal-most stent segment 32 within the sheath 25 when the expandable member 24 is inflated. Such spacing is created, as shown in FIG. 5C, by releasing force against the pusher tube 86 and retracting both the pusher tube 86 and the sheath 25 a short distance simultaneously. The engagement of the valve member 58 with the stent segments 32 moves those stent segments 32 within the sheath 25 away from those stent segments 32 distal to the sheath 25. The length of this spacing is preferably equal to the length of about ½-1 stent segment, e.g., in one embodiment about 2-4 mm. By observing the radiopaque marker 56 on the sheath 25, the operator can adjust the spacing to be suitable in comparison to the length of the marker 56, which preferably has a length equal to the desired spacing distance.

The expandable member 24 is then inflated by delivering inflation fluid through the inflation lumen 66, as shown in FIG. 5D. The exposed distal portion of the expandable member 24 expands so as to expand the stent segments 32 thereon into engagement with the lesion L. If predilatation was not performed, the lesion L may be dilated during the deployment of the stent segments 32 by appropriate expansion of the expandable member 24. The sheath 25 constrains the expansion of the proximal portion of the expandable member 24 and those stent segments 32 within the sheath 25.

The expandable member 24 is then deflated, leaving the stent segments 32 in a plastically-deformed, expanded configuration within the lesion L, as shown in FIG. 5E. With the stent segments 32 deployed, the expandable member 24 may be retracted within the sheath 25, again maintaining force against the pusher tube 86 to slide the stent segments 32 toward the distal end of the expandable member 24. The expandable member 24 is moved proximally relative to the stent segments 32 until the distal-most stent segment engages the stop 78, (see FIGS. 2A-2B), thereby placing the stent segments 32 in position for deployment. The stent delivery catheter 20 is then ready to be repositioned at a different lesion in the same or different artery, and additional stent segments may be deployed. During such repositioning, the guidewire tube 34 facilitates smooth tracking over the guidewire GW. Advantageously, multiple lesions of various lengths may be treated in this way without removing the stent delivery catheter 20 from the patient's body. Should there be a need to exchange the stent delivery catheter 20 with other catheters to be introduced over the guidewire GW, the guidewire tube 34 facilitates quick and easy exchanges.

During practice of the foregoing processes, it is advantageous for the user of the stent delivery catheter to have the ability to ascertain the number of stent segments deployed at each lesion and the number of stent segments remaining in the delivery catheter following each deployment. Alternatively, or in addition, it is also advantageous for the user to have the ability to ascertain the distance that the outer sheath has been withdrawn or advanced relative to the inner shaft 27 and/or the pusher tube 86. As noted in the description of the procedures above, fluoroscopy may be used to view the number of stent segments that have been deployed following inflation of the expandable member 24. Radiopaque markers attached to the stent segments 32 enhance such visualization. However, in some circumstances, fluoroscopic imaging does not provide sufficient clarity to accurately discern the number of stent segments that are being (or have been) deployed from the delivery catheter, or the distance that a first portion of the catheter has been translated relative to another portion of the catheter. The mechanisms and methods described below in relation to FIGS. 7-14 are intended to provide the capability to count the stent segments that are exposed for deployment during the paving process prior to balloon inflation, and/or to determine the relative spacing between the outer sheath and the inner shaft and/or pusher tube. Alternatively, the mechanisms and methods are intended to provide the capability to count the number of stent segments that are deployed following balloon inflation, and/or that remain in the delivery catheter following each deployment. Other and further functions and advantages will be understood after consideration of the descriptions below.

Turning to FIGS. 7A-B, a pair of embodiments of a stent delivery catheter having an optical fiber counter mechanism are shown. The stent delivery catheter 20, the distal end of which is shown in the Figures, is generally as described above, having an outer sheath 25 slidably disposed over an inner shaft. An expandable member 24, preferably an inflatable balloon, is mounted to the inner shaft and is exposed by retracting the sheath 25 relative to the inner shaft. A tapered nosecone 28 is mounted distally of the expandable member 24. A plurality of stent segments 32 are disposed on the expandable member, and are biased distally by a pusher tube 86. A guidewire tube 34 extends through the center of the distal end of the catheter.

Turning first to FIG. 7A, an optical fiber 202 is formed integrally with the pusher tube 86. Alternatively, the optical fiber 202 may be attached to the external surface of the pusher tube 86, or it may be located in a groove or other location on the external surface of the pusher tube 86. The optical fiber 202 is capable of carrying both a transmission signal (for transmitting out of the distal end of the optical fiber) and a received signal (that is detected by the optical fiber and transmitted back to the user interface). This is represented schematically by the arrows “T” and “R” in the Figures. The distal end of the optical fiber 202 is located at or near the distal end of the pusher tube 86, and is provided with a light transmission area 204 that is oriented such that it directs a light beam transmitted by the optical fiber outward, toward the outer sheath 25. The optical fiber 202 is of conventional construction and has a size and shape that permits it to be formed with or attached to the pusher tube 86. The optical fiber 202 is capable of transmitting a light beam from its proximal end to the transmission area 204, then receiving a reflected beam and transmitting it back to the proximal end, where it may be processed by a suitable user interface mechanism 205. (See FIG. 1). The user interface mechanism 205 may be wholly or partially contained in the handle, as shown in FIG. 1, or it may be a separate component not associated with the handle.

A plurality of reflective strips 206 are located on the outer sheath 25. The reflective strips 206 are oriented such that the reflective portions of the strips are directed radially inward to allow reflection of the light beam transmitted by the transmission area 204 of the optical fiber 202 when the transmission area 204 encounters each of the reflective strips 206. The reflective strips 206 may be molded or formed into the body of the outer sheath 25, or they may be attached or otherwise affixed to the internal or external surface of the outer sheath 25. Preferably, the reflective strips 206 are spaced apart at regular intervals over the length of at least a portion of the outer sheath 25. For example, the reflective strips 206 may be spaced apart a distance that is the same as, or that is a fraction of, the length of an individual stent segment 32.

As the outer sheath 25 is retracted proximally relative to the pusher tube 86, the transmission area 204 of the optical fiber 202 will encounter each successive reflective strip 206. The light beam transmitted by the optical fiber will thereby encounter an alternating pattern of reflection and non-reflection, which is carried as a signal back to the user interface mechanism 205 to indicate the number of reflective strips 206 that have been encountered during the retraction process. Because the distance between each reflective strip is known, the distance that the outer sheath 25 has been retracted relative to the pusher tube 86 may be determined and displayed or otherwise communicated to the user. In this way, the optical fiber mechanism provides information to the user indicating the relative movement of the outer sheath 25, which optionally may be converted into an indication of the number of stent segments and/or the length of the balloon or stent segments that have been exposed or deployed by the delivery catheter.

The user interface mechanism 205 preferably includes a light source equipped to generate the light beam signals transmitted by the optical fiber 202, a receiver for receiving the returned light signal, a processor to analyze the signal and provide information in a format suitable for display to the user, and a suitable display for displaying the processed information. As noted, this may include electronic equipment mounted on or in the handle 38, or it may be wholly or partially contained in a separate unit from the handle. Those skilled in the art will recognize the types of electronic equipment that are suitable for providing the foregoing functions and features of the user interface 205.

FIG. 7B shows an alternative embodiment in which the optical fiber 202 is formed into or attached to the tubular leg 72 and/or the inner shaft 27 portion of the delivery catheter. As shown there, the transmission area 204 of the optical fiber is oriented such that it directs a light beam transmitted by the optical fiber outward, toward the outer sheath 25, where the light beam encounters a plurality of reflective strips 206. Thus, as the outer sheath 25 is retracted relative to the tubular leg 72 or the inner shaft 27, the transmission area 204 of the optical fiber 202 encounters each successive reflective strip 206 formed on or in the outer sheath 25. As with the previous embodiment, this information is transmitted back to the user interface mechanism, where it is translated to a measurement of distance or number of stent segments.

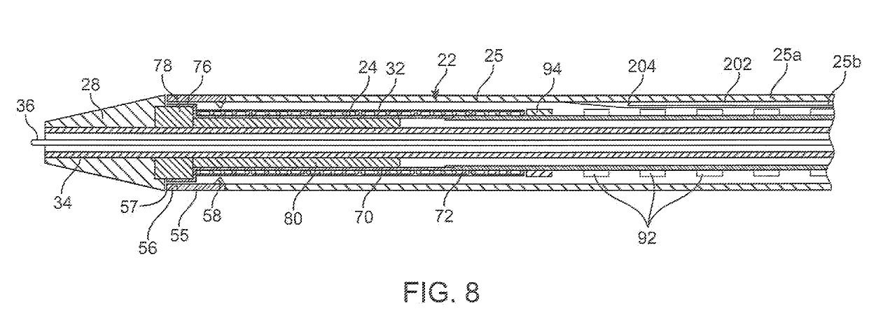

Turning next to FIG. 8, there is shown an embodiment of a stent delivery catheter having an optical fiber 202 formed integrally with or attached to the inner surface of the outer sheath 25. The optical fiber 202 may be of any of the types described above in relation to FIGS. 7A-B. In the embodiment shown, the optical fiber 202 is embedded into the internal surface of an outer layer 25a of the outer sheath 25, and an inner layer 25b is disposed over the internal surface of the outer layer 25a to effectively seal the optical fiber 202 between the outer layer 25a and the inner layer 25b of the sheath 25. In the preferred embodiment, the outer layer 25a of the sheath comprises a polymeric material such as Pebax having a stainless steel reinforcing braid embedded therein. The inner layer is preferably PTFE, or a similar low-friction polymeric material.

The transmission area 204 of the optical fiber is oriented such that it directs a light beam transmitted by the optical fiber inward, toward the pusher rod 86, where the light beam encounters the cylindrical rings 92 of the pusher tube 86. Thus, as the outer sheath 25 is retracted relative to the pusher tube 86, the transmission area 204 of the optical fiber 202 encounters each successive cylindrical ring 92 formed on the pusher tube 86. The pusher tube 86 is preferably made of a material having some degree of reflectivity, such as the materials described above, thereby providing the ability for the optical fiber 202 to transmit location information based on the light reflected by the cylindrical rings 92. As with the previous embodiment, this information is transmitted back to the user interface mechanism 205, where it is translated to a measurement of distance or number of stent segments.

In optional embodiments not shown in the Figures, the optical fiber 202 may be carried on or in the outer sheath 25 and positioned such that the light transmission area 204 reflects a light beam off the strut portions forming the stent segments 32, or reflect (or not reflect) off markers or other features provided on the stent segments 32. Thus, the optical fiber 202 is able to transmit information relating to the number of stent segments 32 that have moved past the light transmission area 204. This information is transmitted back to the user interface mechanism 205 for display to the user.

The optical fiber mechanisms described above in relation to FIGS. 7A-B and 8 utilize an optical fiber that is preferably embedded or connected to a first component of the stent delivery catheter. The optical fiber then interacts with one or more other components of the stent delivery catheter to provide an independent measure of a distance or of a number of fixed elements that have moved through a transmission area path when the separately movable portions of the delivery catheter are moved relative to one another. In the embodiments described above, the one or more other components of the device include either reflective strips, portions of the stent segments, or portions of the pusher tube. It is also contemplated that other members may be used to provide the interaction with the optical fiber used to determine the distance or number measurement.

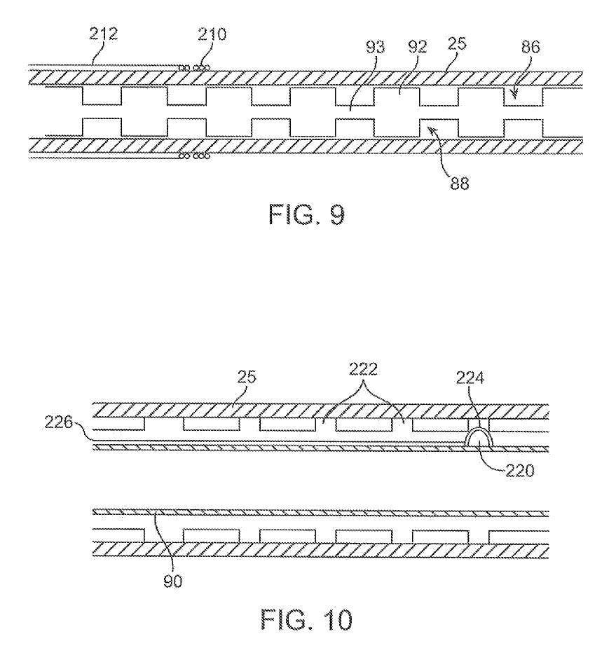

Turning next to FIG. 9, another distance measurement or counting mechanism is illustrated. In particular, FIG. 9 is a cross-sectional illustration of a portion of the stent delivery catheter coinciding with the distal extension 88 of the pusher tube 86. The outer sheath 25 surrounds the distal extension 88, which comprises a plurality of cylindrical rings 92 connected together by longitudinal connectors 93. A resonating wire coil 210 is attached to the exterior of the outer sheath 25 at a selected position, preferably a position proximal of the location of the expandable member 24 such that the distal extension 88 extends through the portion of the outer sheath 25 to which the wire coil 210 is attached. Alternatively, the wire coil 210 may be embedded within the outer sheath 25, or attached to the internal surface of the outer sheath 25. The wire coil 210 includes a plurality of circumferential coils formed over a section of the outer sheath 25. A pair of wire leads 212 extend proximally from the wire coil 210 to the proximal end of the delivery catheter, where they are connected to a user interface mechanism 205. (See FIG. 1). Thus, the wire coil 210 and wire leads 212 define a continuous loop of wire.

A voltage is applied to the wire making up the wire coil 210 and the wire leads 212, creating a resonance amplitude that is measured and transmitted in a useful form to the user interface mechanism 205. The voltage may be created by use of a battery or any other suitable source of electricity, and the voltage may be measured using any appropriate voltage measuring device, preferably one incorporated in the user interface mechanism 205.

When the stent delivery device is being used, the outer sheath 25 is retracted or advanced relative to the pusher rod 86 during the processes described above in relation to FIGS. 5A-E. As this movement occurs, the cylindrical rings 92 of the pusher rod 86 pass successively through the wire coil 210. Because the pusher rod 86 is formed of stainless steel or other conductive material, the passage of alternating cylindrical rings 92 and blank areas creates a measurable detuning of the resonance and thus a measurable decrease in the voltage in the wire coil 210 that can be measured. This voltage decrease is detected and is used to increment a counter corresponding to the number of cylindrical rings 92 that have passed through the wire coil 210. Because this is a known distance, the measurement is able to be used to determine the position of the outer sheath 25 relative to the pusher 86, a distance that indicates the position of the stent valve 58 with respect to the stent segments 32 during the paving process. In addition, the detection mechanism will indicate a potential problem in the event that the outer sheath 25 does not move relative to the pusher 86 when it is intended to do so.

Turning next to FIG. 10, yet another distance measurement or counting mechanism is illustrated. In particular, FIG. 10 is a cross-sectional illustration of a portion of the stent delivery catheter coinciding with the location of the proximal portion 90 of the pusher tube and having a pressure sensing member affixed thereon. The outer sheath 25 surrounds the pusher tube 90. The location shown in FIG. 10 may be at any suitable location on the length of the catheter body 22, but it is preferably at a location near to the distal end of the catheter body. A bump 220 of an elastomeric material is attached to the pusher tube 90. The elastomeric material may be a silicone, a polyurethane, or any suitable material having suitable elastomeric properties. A variable resistivity ink (VRI) 224 is coated over the bump 220. An optional additional layer of parylene may also be coated over the variable resistivity ink. A pair of electrical leads 226, such as lead wires, extend over the external surface of the pusher rod 90 to the proximal end of the delivery device, where they connect to the user interface mechanism 205 (see FIG. 1). The user interface mechanism 205 preferably includes a power supply to apply a voltage across the electrical leads 226, an amplifier board to facilitate the detection of changes in the resistance of the variable resistivity ink, and a suitable display to show when resistivity changes are encountered.

The outer sheath 25 includes a plurality of circumferential marker bands 222 spaced at regular intervals along a portion of the length of the outer sheath 25. Preferably, the marker bands 222 are spaced apart a distance that is the same as or otherwise comparable to the length of the stent segments 32 being deployed. Each marker band 222 comprises a slight indentation formed on the internal surface of the outer sheath 255, which creates a void space between the outer sheath 25 and the pusher tube 90.

As will be appreciated by reference to FIG. 10, as the outer sheath 25 is moved proximally or distally relative to the pusher rod 90, the elastomeric bump 220 encounters an alternating pattern of first being compressed between the outer sheath 25 and the pusher rod 86, then encountering the marker bands 222 in which the elastomeric bump 220 is able to expand to its normal size and shape. The significance of this alternating pattern is that the resistivity of the ink coated on the elastomeric bump changes as the elastomeric bump 220 is alternatingly compressed and expanded. For example, for a conventional VRI, the resistivity will be as high as 10 Megohms per square millimeter when the VRI is not being compressed, but sill drop to as low as 300 Kohms per square millimeter under maximum pressure. This resistivity change is measured by applying a voltage to the pressure sensor and sensing the alternating changes as the outer sheath 25 moves relative to the pusher rod 90.

Accordingly, as the outer sheath 25 is moved relative to the pusher rod 86, such as during the paving and resetting steps described above, the resistivity of the ink on the elastomeric bump 220 changes as the elastomeric bump encounters each of the marker bands 222 formed on the internal surface of the outer sheath 25. This effect is detected, measured, and then suitably displayed on the user interface mechanism 205 to provide information to the user concerning the amount of relative movement between the outer sheath 25 and the pusher rod 90, of the number of stent segments that have been exposed by retraction of the outer sheath 25, or the number of stent segments that remain covered by the sheath 25, or other suitable information able to be determined from the distance measurements detected and recorded by the mechanism.

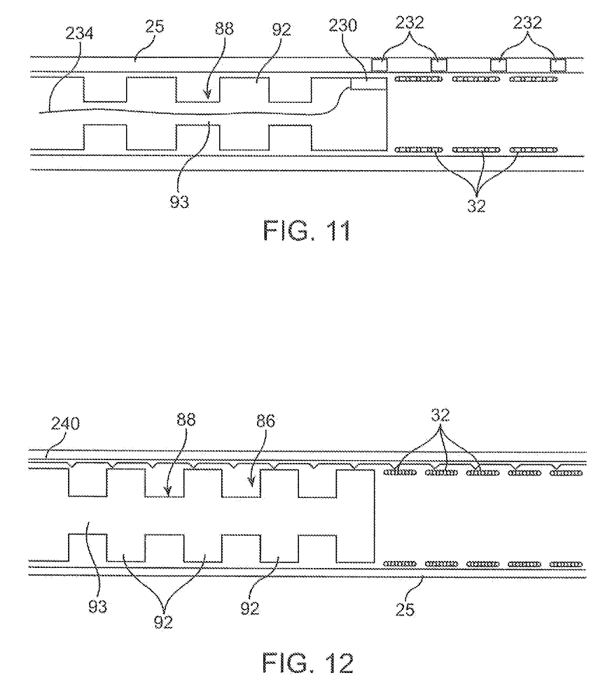

Turning next to FIG. 11, another alternative counting and/or movement measuring mechanism includes at least one sensor 230 that is mounted on or attached to the distal end of the pusher rod 86. A lead 234, such as an electrically conductive wire, is attached to each sensor 230 and is routed proximally to the user interface mechanism 205 on the handle 38. A plurality of suitable markers 232 are embedded in or otherwise attached to the outer sheath 25 and are spaced at regular, known intervals. Preferably, the markers 232 are spaced apart a distance that is the same as, or comparable to, the length of one or more of the stent segments 32 carried by the stent delivery catheter.

In a preferred embodiment, the sensor 230 is a microsized bare die hall effect sensor having a size small enough to be mounted on or attached to the distal end of the pusher rod 86. The hall effect sensor 230 is adapted to sense magnetic field sources. Accordingly, in the preferred embodiment, the markers 232 each include a material or particles of a material having magnetic properties. The hall effect sensor 230 thereby generates and transmits an electronic signal indicating each time the sensor 230 encounters a marker 232 as the outer sheath 25 is moved relative to the pusher rod 86.

In alternative embodiments, the sensor 230 may be mounted or attached to another portion of the delivery catheter, such as the inner shaft 27, the tubular leg 72, the guidewire tube 34 extending beneath the expandable member 24, the outer sheath 25, or another suitable location. The markers 232 may also be mounted or attached to one of these other portions of the delivery catheter, provided that they are able to interact operatively with the sensor 230.

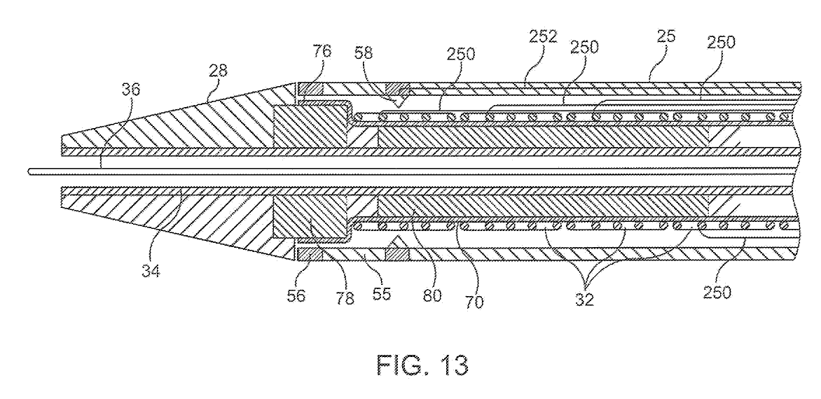

The mechanisms illustrated in FIGS. 12 and 13 utilize one or more electrical contacts that generate an electrical signal that may be translated to a distance or number measurement. Turning first to FIG. 12, a portion of the stent delivery catheter is shown in cross-section. The distal extension 88 of the pusher rod 86 is shown, with the pusher rod engaging the proximal-most of the stent segments 32. The outer sheath 25 surrounds the pusher rod 86 and the stent segments 32. The outer sheath 25 includes a stainless steel or other metallic braid 240 that is embedded within the outer sheath 25, such as between an inner polymeric layer and an outer polymeric layer. In the embodiment shown, the reinforcing braid 240 is formed of a least one insulated wire. The braid may optionally include a plurality of such insulated wires, or a single such wire braided with other non-conductive members. The insulation is removed from an inward-facing conductive portion of the braid 240 at regular intervals along the outer sheath 25, thereby exposing the braid 240 to the cylindrical rings 92 forming the distal extension 88 of the pusher rod 86. The exposed inward-facing portions of the braid are preferably raised inwardly from the wall of the sheath 25 so as to contact the rings 92 of the pusher rod 86.

A low-voltage electric circuit is then constructed between the braid 240 and the pusher rod 86. For example, the user interface mechanism 205 may be provided with a battery or other voltage source that is applied to the pusher rod 86 and to the braid 240 at each of their proximal ends. A current measuring device, also preferably contained in the user interface mechanism 205, is coupled to each of the pusher rod 86 and the braid 240. Accordingly, when a cylindrical ring 92 of the pusher rod 86 engages the portion of the braid 240 having the insulation removed from it, a circuit is formed between the conductive braid 240 and the conductive pusher rod 86, which will cause a measurable current to flow and be measured by the current measuring device. When the exposed portion of the braid 240 is not in contact with the cylindrical ring 92, no circuit is present and no current is measurable.

Accordingly, because the distance between cylindrical rings 92 is known, and because the distances between the exposed portions of the braid 240 are known, the amount of movement of the outer sheath 25 relative to the pusher rod 86 can be determined by monitoring the number of times a measurable current is generated in the foregoing circuit when the outer sheath 25 is moved. This determination may be translated into either a distance measurement, or correlated to a numerical measure of the number of stent segments that have been exposed, and displayed to the user at the user interface mechanism 205.

In alternative embodiments, rather than exposing portions of the braid 240 for an electrical contact, pressure sensitive switches may be located at regular intervals on the internal surface of the outer sheath 25. The switches are electrically isolated from the patient by the insulating layer on the outer sheath 25, but are electrically connected by leads to the user interface mechanism 205 on the handle (or elsewhere). The switches are preferably located such that they are activated by the pressure encountered when the cylindrical rings 92 pass through the portion of the outer sheath 25 carrying the switch.

Turning next to FIG. 13, the stent delivery catheter includes an electrically conductive wire 250 releasably attached to each stent segment 32. Each wire 250 is formed of an electrically conductive material, such as a metallic strip or fiber such as tungsten wire. Each wire 250 extends proximally to the handle 38 where it is separately manipulatable and where it is electrically connected to the user interface mechanism 205. The stent valve 58 is also formed of or is provided with an electrically conductive material that is connected by a lead 252 to the user interface mechanism 205. The stent valve 58 is oriented such that it contacts the stent segment wire 250 connected to the stent segment 32 underlying the stent valve 58 to form a circuit that is able to be monitored by application of a voltage provided at the user interface mechanism 205. Accordingly, the user is able to determine which stent segment 32 is located directly beneath the stent valve 58, and thereby know the number of stent segments that are exposed distally of the outer sheath 25.

As the stent segments 32 are deployed, the wires 250 are decoupled from the stent segments 32 by being severed or otherwise released as the stent segments 32 expand. The decoupled wires 250 may then be retracted by exerting traction on the wires through a suitable mechanism on the handle 38.

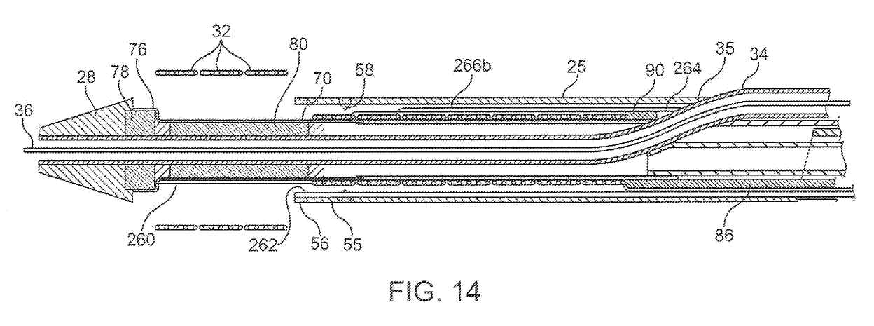

Turning next to FIG. 14, another alternative mechanism for determining and providing distance or number information to the user includes provision of one or more position wires that are attached to one or more of the components of the distal end of the delivery catheter and that extend proximally to the handle. As a first example, FIG. 14 shows a position wire 260 attached to the stent stop 78 near the distal end of the delivery device. The position wire 260 extends proximally to the handle 38 beneath the column of stent segments 32 carried by the inner shaft so as not to interfere with the deployment of the stent segments 32. As the outer sheath 25 is withdrawn proximally relative to the inner shaft 27, the position wire 260 will remain in a fixed position relative to the inner shaft 27 and the components that are fixed in relation to the inner shaft 27, but the position wire 260 will change position relative to the outer sheath 25 and the components that are fixed in relation to the outer sheath 25.

Preferably, multiple position wires are deployed. For example, an additional position wire 262 may be fixed to a portion of the garage 55. Still another position wire 264 may be fixed to the pusher rod 86. Additional position wires 266a-n may be fixed to each individual stent segment 32, or selected ones of the stent segments 32. Each such position wire extends proximally to the handle 38, preferably through a common lumen formed in the proximal portion of the delivery catheter. The proximal end of each position wire is preferably fixed with an index or other indicator to determine a baseline relative position between each of the position wires. Then, as the individual stent delivery catheter components are moved relative to one another, the relative motion may be monitored and the positions of the components determined by reference to the positions of the position wires 260, 262, 264. This information may be viewed directly by the user, or it may be translated into a distance measure or number measure and displayed to the user by an appropriate display contained on the user interface mechanism 205.

The position wires each preferably comprise a material that is strong, relatively lightweight, and that exhibits minimal elongation under axial tension so as to maintain a consistent length when exposed to the conditions expected during catheterization and other procedures. A particularly preferred material is a tungsten wire. Other materials and structures may also be used, such as fibers, filaments, or the like.

The foregoing descriptions of the preferred embodiments are intended to serve as non-limiting examples of the devices and methods of the present invention. Variations of the devices and methods described herein have also been contemplated. For example, it should be understood that when the movement of the pusher tube, sheath, or stent segments is described in relation to other components of the delivery catheter, such movement is relative and will encompass both moving the sheath, pusher tube, or stent segments while keeping the other component(s) stationary, keeping the sheath, pusher tube or stent segments stationary while moving the other component(s), or moving multiple components simultaneously relative to each other. In addition, in any of the above embodiments that include electrical conductors, light energy conductors, or the like, these conductors may be incorporated into the device by embedding in the body of a component of the delivery catheter, attachment to the internal or external surface of such a component, or by other suitable means. Still further, electrical conduction may be obtained through use of copper wire or other suitable conductor (with insulation if appropriate) that may be incorporated into the reinforcing braid embedded in the wall of the outer sheath in the preferred embodiments (i.e., all or some of the strands of the reinforcing braid may be made of a suitably conductive material and used as an electrical conductor). Still other variations are possible.

While the foregoing description of the invention is directed to a stent delivery catheter for deploying stents into vascular lumens to maintain patency, it should be understood that various other types of wire-guided catheters also may embody the principles of the invention. For example, balloon catheters for angioplasty and other purposes, particularly those having a slidable external sheath surrounding the balloon, may be constructed in accordance with the invention. Other types of catheters for deployment of prosthetic devices such as embolic coils, stent grafts, aneurism repair devices, annuloplasty rings, heart valves, anastomosis devices, staples or clips, as well as ultrasound and angiography catheters, electrophysiological mapping and ablation catheters, and other devices may also utilize the principles of the invention.

Although the above is complete description of the preferred embodiments of the invention, various alternatives, additions, modifications and improvements may be made without departing from the scope thereof, which is defined by the claims.

What is claimed is:

1. A catheter comprising:
an elongated flexible shaft having a distal end and a proximal end, said flexible shaft including an outer sheath and an inner shaft,
an actuator at the proximal end of the shaft, said actuator configured to move said outer sheath proximally relative to said inner shaft from a first position in which at least a first portion of said inner shaft is covered by said outer sheath to a second position in which the first portion of said inner shaft is not covered by said outer sheath, and
an optical fiber attached to said flexible shaft, said optical fiber being configured to detect a parameter related to said movement of said outer sheath from said first position to said second position.

2. The catheter of claim 1, wherein said parameter is the distance the outer sheath moves relative to the inner shaft.

3. The catheter of claim 1, further comprising a plurality of prostheses carried on said inner shaft, and wherein said parameter is the number of prostheses that pass a portion of said optical fiber.

4. The catheter of claim 1, further comprising a sensor for receiving light from said optical fiber, said sensor being disposed on at least one of said outer sheath or said inner shaft.

5. The catheter of claim 3, further comprising a sensor for receiving light from said optical fiber, said sensor being disposed on at least one of said prostheses.

6. The catheter of claim 1, wherein said optical fiber is attached to said outer sheath.

7. The catheter of claim 6, wherein said optical fiber transmits light, the catheter further comprising a marker configured to reflect or absorb the light when the marker is aligned with the optical fiber.

8. The catheter of claim 7, wherein said flexible shaft further comprises a pusher member, and wherein said marker is disposed on said pusher member.

9. The catheter of claim 7, wherein said marker is attached to said inner shaft.

10. The catheter of claim 1, wherein said flexible shaft further comprises a stent positioned over said inner shaft, and wherein said optical fiber detects said stent.

11. The catheter of claim 10, wherein said stent comprises a plurality of stent segments.

12. The catheter of claim 11, wherein said parameter comprises the number of stent segments passing in front of the optical fiber.

13. The catheter of claim 11, wherein said parameter comprises a strut on each stent segment.

14. The catheter of claim 1, wherein said optical fiber is attached to said inner shaft.

15. The catheter of claim 14, wherein said optical fiber transmits light, the catheter further comprising a marker configured to reflect or absorb the light when the marker is aligned with the optical fiber.

16. The catheter of claim 15, wherein said flexible shaft further comprises a pusher member, and wherein said marker is disposed on said pusher member.

17. The catheter of claim 15, wherein said marker is attached to said outer sheath.

18. The catheter of claim 15, wherein said flexible shaft further comprises a stent positioned over said expandable member.

19. The catheter of claim 18, wherein said stent comprises a plurality of stent segments.

20. The catheter of claim 1, wherein said flexible shaft further comprises a pusher member, and wherein said optical fiber is attached to said pusher member.

21. The catheter of claim 20, wherein said optical fiber transmits light, the catheter further comprising a marker configured to reflect or absorb the light when the marker is aligned with the optical fiber.

22. The catheter of claim 21, wherein said marker is attached to said outer sheath.

23. The catheter of claim 20, wherein said flexible shaft further comprises a stent positioned over said inner shaft.

24. The catheter of claim 23, wherein said stent comprises a plurality of stent segments.

25. The catheter of claim 1, further comprising an expandable member disposed on said inner shaft at or near a distal end thereof.

26. A catheter comprising:
an elongated flexible shaft having a distal end and a proximal end, said flexible shaft including an outer sheath and an inner shaft,
an actuator at the proximal end of the shaft, said actuator configured to move said outer sheath proximally relative to said inner shaft from a first position in which at least a first portion of said inner shaft is covered by said outer sheath to a second position in which the first portion of said inner shaft is not covered by said outer sheath, and
a sensor attached to said flexible shaft at or near its distal end, said sensor configured to detect a parameter related to said movement of said outer sheath from said first position to said second position.

27. The catheter of claim 26, wherein said parameter is the distance the outer sheath moves relative to the inner shaft.

28. The catheter of claim 26, further comprising a plurality of prostheses carried on said inner shaft, and wherein said parameter is the number of prostheses that pass a portion of said optical fiber.

29. The catheter of claim 26, wherein said sensor comprises a resonating wire coil.

30. The catheter of claim 29, further comprising a lead wire connected to said resonating wire coil and extending proximally to the proximal end of said flexible shaft.

31. The catheter of claim 29, wherein a resonance amplitude of said wire coil changes when said outer sheath moves from said first position to said second position.

32. The catheter of claim 29, wherein said flexible shaft further comprises a plurality of spaced members, and wherein a resonance amplitude of said wire coil changes when each of said spaced members passes through said wire coil.

33. The catheter of claim 32, wherein said plurality of spaced members are disposed on the inner shaft.

34. The catheter of claim 32, further comprising a pusher rod, and wherein said plurality of spaced members are disposed on said pusher rod.

35. The catheter of claim 26, wherein said sensor comprises a pressure sensor including a material having electrical resistivity that is pressure dependent.

36. The catheter of claim 35, wherein said material comprises a variable resistivity ink.

37. The catheter of claim 35, wherein said pressure sensor comprises a first elastomeric member and a coating of said material on said first elastomeric member.

38. The catheter of claim 37, wherein said flexible shaft further comprises a pusher member in a sliding engagement within said outer sheath, and wherein said elastomeric member is attached to an external surface of said pusher member.

39. The catheter of claim 38, further comprising a plurality of bands formed on an internal surface of said outer sheath, said bands each adapted to engage said sensor and to thereby change the pressure applied by said outer sheath to said sensor in relation to a condition in which a band does not engage said sensor.

40. The catheter of claim 39, further comprising a conductive lead attached to said sensor and extending to the proximal end of said flexible shaft.

41. The catheter of claim 26, wherein said sensor comprises a hall effect sensor and wherein said catheter further comprises a plurality of magnetic markers attached at intervals over a portion of the flexible shaft near its distal end.

42. The catheter of claim 41, wherein said sensor is attached to said inner shaft and said markers are attached to said outer sheath.

43. The catheter of claim 41, wherein said sensor is attached to said outer sheath and said markers are attached to said inner shaft.

44. The catheter of claim 41, wherein said markers are spaced at regular intervals corresponding to the length of each of a plurality of stent segments carried by the expandable member.

45. The catheter of claim 41, further comprising a pusher rod, and wherein said sensor is attached to said outer sheath and said markers are attached to said pusher rod.

46. The catheter of claim 41, further comprising a pusher rod, and wherein said sensor is attached to said pusher rod and said markers are attached to said outer sheath.

47. The catheter of claim 26, further comprising an expandable member disposed on said inner shaft at or near a distal end thereof.

48. A catheter comprising:
an elongated flexible shaft having a distal end and a proximal end, said flexible shaft including an outer sheath and an inner shaft,
an actuator at the proximal end of the shaft, said actuator configured to move said outer sheath proximally relative to said inner shaft from a first position in which at least a first portion of said inner shaft is covered by said outer sheath to a second position in which the first portion of said inner shaft is not covered by said outer sheath, and
a plurality of electrically conductive members, said plurality of electrically conductive members configured to detect a parameter related to said movement of said outer sheath from said first position to said second position.

49. The catheter of claim 48, wherein said parameter is the distance the outer sheath moves relative to the inner shaft.

50. The catheter of claim 48, further comprising a plurality of prostheses carried on said inner shaft, and wherein said parameter is the number of prostheses that pass a portion of said optical fiber.

51. The catheter of claim 48, wherein said elongated flexible shaft further comprises a pusher member, and said plurality of electrically conductive members comprises a first member attached to said outer sheath and a second member comprising said pusher member or disposed on said pusher member.

52. The catheter of claim 51, wherein said first member comprises a reinforcing braid embedded in said outer sheath.

53. The catheter of claim 52, wherein said reinforcing braid has a plurality of exposed regions spaced at intervals along the outer sheath.

54. The catheter of claim 52, wherein said pusher member comprises a plurality of spaced members that slidably engage the internal surface of the outer sheath.

55. The catheter of claim 54, wherein said plurality of spaced members comprise a plurality of cylindrical rings.

56. The catheter of claim 48, wherein said plurality of electrically conductive members comprises a first member attached to said outer sheath and a second member attached to a stent carried by said expandable member.

57. The catheter of claim 56, wherein said first member comprises an electrically conductive lead attached to a contact on said outer sheath.

58. The catheter of claim 56, wherein said second member comprises an electrically conductive lead releasably attached to said stent.

59. The catheter of claim 58, wherein said stent comprises a plurality of stent segments, and wherein each stent segment includes an electrically conductive lead releasably attached thereto.

60. The catheter of claim 48, further comprising an expandable member disposed on said inner shaft at or near a distal end thereof.

61. A catheter comprising:
an elongated flexible shaft having a distal end and a proximal end, said flexible shaft including an outer sheath and an inner shaft,
an actuator at the proximal end of the shaft, said actuator configured to move said outer sheath proximally relative to said inner shaft from a first position in which at least a first portion of said expandable member is covered by said outer sheath to a second position in which the first portion of said expandable member is not covered by said outer sheath, and
a plurality of position indicator members, said plurality of position indicator members configured to detect a parameter related to said movement of said outer sheath from said first position to said second position.

62. The catheter of claim 61, wherein said parameter is the distance the outer sheath moves relative to the inner shaft.

63. The catheter of claim 61, further comprising a plurality of prostheses carried on said inner shaft, and wherein said parameter is the number of prostheses that pass a portion of said optical fiber.

64. The catheter of claim 61, wherein said plurality of position indicator members includes a first position wire attached to the outer sheath at or near its distal end.

65. The catheter of claim 64, wherein said plurality of position indicator members includes a second position wire attached to the inner shaft at or near its distal end.

66. The catheter of claim 64, wherein said flexible shaft further comprises a pusher, and wherein said plurality of position indicator members includes a third position wire attached to said pusher.

67. The catheter of claim 65, wherein said flexible shaft further comprises a pusher, and wherein said plurality of position indicator members includes a third position wire attached to said pusher.
